SEQUENCE LISTING (1 sequence)

SEQ ID NO: 1 - DNA, 4994 bp, Gossypium hirsutum
  misc_feature (112)..(4035): A or C or G or T
tgggtacgggccccccctcgaggtcgacggtatcaataagcttgaccaaatggcttaatcctgatttatgcaagaatttggttctgattcgttttacatgttggttttgatnctttaattcctttttcttgtagttatgttccaaggctgatggttttgactgatttgattcaggttttgactttgtaggtgctttctttggaggaatttgtttggattatttgttgttctactcagggactggagctgagtgtatctgccctttgatactgattcaagttattggtaaaagtcctgccagagttggaataatcgccagcccctntntggttgtggctgaggctgcaattttgattctgtcagcagttgcggtggcactgattcttgggaagtgcattttagatgctcactaatcctgctgtgccatggcttttgtttaatgtacattcgtttganagcaagtcttgcagaaaatggtctcgactgtatgctttctactgcaaaattangtttaatcaaataacttgctaatatctagcggaatatcttatgatgggtagctnctttctnacgagattcagtcattgtgaacaaccatgtaattgtagtacttcttgaacattggaggttctgtgctatnctgaagagggaatttgaagcgttactgcactgtaacttgcatatagtctgaatttctggttgacaaaatggaagctgagggaggaaatgctaaccaccaangaggtgcccttcatcagatgttccctgttgttttacctgttcttctaatatcaattggatatgttgaccctgggaagtgggttgccactgttgaaggaggtgcacggtttggtattgacctggtagttcccatgcttctttttaattgtgctgcaattttgtgtcaatatctgtcagctagaattggtgtggtgactgggcgtggtcttgctcaggtattctatacacgcttgtgcatnntacaagaataaaaagattaggaattattctgtttgtaagacaatttctttgacaactataatactgatctgtgtacttgtagaacnctataaaaaataacaacatgacttagttaaattctcnanagttgtttaattatatttctgcctagattaaaaagcataagaactccttttttacaaatgcangcttattaatcttttataaagtgcttatatataaccagaatgtcatgtgcaaaggtgcttgtgcttatttgtggacttgtgttttaccctttgcatgaaacatatatatttcttaatttatggcgatagatttgcagcgaagagtatgacaagtccactcgtttattccttggagttcaagcagagctttctgtggttgcgttggaccttaccatggtagctataattctttttatttgcacttaatatagtgatatttgttctattctcatgaaaaatgtctatattttcccaggtcctgggngccgcacatgggattaatcttctcttcggggtggatctctccactggtgtttttctagctgctcttgatgctgttttattcccagtttttgcctccaccttggtatgttaaactgctttgttttattgcacaatgtctcccttgcaacatcatagcagtctaaacaattttcaacattaggatcactgcagggcaagtttcctatgcatctatgcagcaggtttcatattgctttcttatgtttttggagtgctcctcagtcaaccagaaatttctatttccatgcttgggatgccaacaaagttgagtggggagagtgcatttgccctgatgagtcttcttggagcaagcatcatgcctcacaatttttatctacattcttctattgttcaggtatctgcgattggacttgtatatgcatgtatgtacatgagtcttgtatatgggcctttatatttcagttttaatggaggggggactcctgtattatctgtaaatttatttcaactaagaatttgacattttctttgtcaatacttggattttagaagcctctttaggatctttgcatgtggatgctgtccttgtagattcactacaatctgagtcaaatgcatgtataattttcctcaattagtggtcttctatagttatgacattgttctcaaatatagggtcggctgatgttcattgcaccatgcttaaatgttatggggcatacaattcttgaactgcgggacaaattggtgcaccttatatcttttatgcttgcaatatcatttggtttttttttatggtcctgcctttttactttttggagttcaattttatgctattcattctcaactctctgaagttttgttcttctgtccacaggagcatctgggaccaccaaatacttctaagagtgccttatgtcacaaccatctttttgccatcttaggcgtctttggtggaatttatctagtaaattatgtgctgatgaattcagctgcaaatgtgttctacaatgctggccttgtcttggttacttttcatgatgcaatggagcaggttaatttctttcgcttttctgataagctattgtttatgatggctttcaagaatcatgtttaaatatttctcaacaattgagttaatatgtggatattaataaattccttttggaaatgattaaaacaggtattcaggaatggtatactgcccctagtctttttgttggttatgttcttatctaatcaacttactgcatcaacctggaatcttggcgggcaagttgtcttgcataatttccttgggcttgacataccgggttggcttcatcgtgcaacaatcaaaattgtagcaattgttccagctctttattgtgtgtggacttctggacctgagggggtataccagatgtttatccttgcgcaggtgatggtagcccttctgttgccatcttcagtgatccccctttttcgggttgcctcatcaagatcaattatgggtgtttacaaagtttctccaattttggagttcctatcactggtaacattcatgggaattctgggtttaaagataatctttgtggtagaaatgatatttgggagtagtgattgggctgggaatttgagattgaatgctgggattagcatgtctgttccttttgttgtgcttctggctactgcttgtgcatcgttttctttgatgctttggctggcagctactcctttaaaatctgccagttctgaaagtaaagctcatgcatggaaatgggatatgaacagaactgtttctgagacagctatagagagagagggaaatgagttaagtgaaactagatattgtggagaggaacctgctcatatacaggaaagatcattagcaccagaaaactccattgaaagtcattcagatttatcttttccaaattataatctggatttgcctgagacaatcatggagtctgagcaggaaatacgtctgacaactgttaatgcgaactcttctagtggtgaatatcctagccccccattctgtggcactgaggaaccagcatccatacctgagttagcttctgctgtagttgatgaggtaacagatgatgtgccgggcacaaagactctgaagattgaatcaatgaactctctggagaaaacagtgagttttgagggagatctgcatatcgaaaaagatgatgatggagattcttgggagcctgaagagccatccaaacctcctggaagtatttcctctttggcaccggatggacctccttcattcaggagcctcagcgggaaaagtgatgatggtggcaatggtactggaagtctctctagattggcaggattaggtcgtgctgcaaggcgtcaattagctgccattcttgatgaattttggggccaattgtatgattttcatgggcaacctacccaagaagcaaaggtgaagaaattagatgtactattgggtgttgattcaaaaccattaaaagtagatacaactgggaaggagtatggtgggtatttcccttcggtgggaggaagaggatctgatgcacttaatggttcaagcctntatgactctccaaagcatttgaagatgcaaaatagtattgatttatcacgtgggtatcctagaggatcatcattgtggtcaagccagatgcaacaattagatgcttatgctcaaaattctagctgtaatgtcatttctggtgaaaggaggtactttagtttgcgtgctgcaccatctgctgaggcatgggattatcaaccagctactgtgcatggatatcagattgcatcttaccttaatcgaattgctaaagatagaagctccaattgtttgaatgatcaaattgaattaccagcatcagattctcctgccatgggtcctacaaattatcgaggctccctggcttctgcattaaggcaaaaatcgcaaaatggggtaactcctgctcagccccctggatttgagaatgttgcagttgctagaagtagtgcactacaatccgaaaggtcttatcatgacaagaacttatctgggataaatgataattctgggatatcagtgaatacaaagaagtatcatagcttaccggacatatctgggctgtctgttcctcatcgagtgcccgagaagagtggccagtgggacagttccattggatatgggttgtccattggtcggacaaattatggaacacccatgtattcaaatgctgggtcaagggtaggtgttccattttcctttgatgagctttctcatttaaagggttatagagatgctttgcctttacagttgggttcaggttctggcactggatccctttggtctagacagccttttgagcagtttggtgtagctgacaaaagtcacactgctggcaatgaagcagttggaagtgggttgaactcagtaactcgggatactgcttctggtgtggatttagagtccaagcttgatatcgaattcctgcagcccgggggatccactagttctagagcggccgccaccgcggtggagctcca

(19) United States
(12) Patent Application Publication
(10) Pub. No.: US 2005/0060772 A1
(43) Pub. Date: Mar. 17, 2005

(54) Regulation of ethylene sensitivity during leaf and flower abscission

(76) Inventors: Joseph Ciardi, Philadelphia, PA (US); Robert J. Cibulsky, Malvern, PA (US)

Correspondence Address: JOHN W. GOLDSCHMIDT, JR. ESQUIRE;DILWORTH PAXON LLP, 3200 MELLON BANK CENTER, 1735 MARKET STREET, PHILADELPHIA, PA, 19103, US

(21) Appl. No.: 10/493,728

(22) PCT Filed: Oct. 28, 2002
(86) PCT No.: PCT/US02/34566

Related U.S. Application Data
(60) Provisional application No. 60/339,596, filed on Oct. 26, 2001.

(51) Int. Cl. A01H 1/00; C12N 15/82

(52) U.S. Cl. 800/287

(57) ABSTRACT

Provided is the tissue specific manipulation of the EIN2 and EIN3 ethylene-signaling genes, permitting the controlled regulation of leaf and/or flower abscission, since flower and leaf abscission are positively regulated by the plant hormone ethylene. In particular, controlled expression of the EIN2 and EIN3, permits reduction of early-season flower abscission and accelerated late-season leaf abscission, preferably under the control of an inducible promoter. When the altered expression of ethylene signaling genes is combined, for example in a preferred embodiment in the cotton plant, both reduced flower abscission and induced premature leaf abscission are achieved, thereby producing a more productive and more efficiently harvested crop. Further provided are new insights into the mechanisms involved in the ethylene signaling pathway.

### REFERENCE TO RELATED APPLICATIONS

This application claims priority to U.S. Provisional Application 60/339,596, filed Oct. 26, 2001, which is herein incorporated by reference in its entirety.

### BACKGROUND OF THE INVENTION

The plant hormone ethylene regulates a wide range of developmental processes, including seed germination, abscission of leaves and flowers, stem elongation, and fruit ripening. Ethylene signal transduction is controlled by a complex multicomponent pathway (Kieber, Annu. Rev. Plant Physiol. Plant Mol. Biol. 48:277-296 (1997). To address the ethylene signaling mechanisms, a molecular/genetic approach has been applied using the ethylene-evoked triple response phenotype of Arabidopsis thaliana seedlings.

In Arabidopsis, the “triple response” typically involves inhibition of root and stem elongation, radial swelling of the stem and absence of normal geotropic response (diageotropism). Etiolated morphology of a plant can be dramatically altered by stress conditions which induce ethylene production, so that, for example, the ethylene-induced triple response provides a seedling with the additional strength required to penetrate compacted soils. Based upon the triple response, a dozen Arabidopsis mutants have been isolated into two classes Ecker, Science 268:667-675 (1995); U.S. Pat. Nos. 5,367,065; 5,444,166; 5,602,322 and 5,650,553, each of which is herein incorporated by reference). One class of mutants, the ein (ethylene insensitive) mutants, showed reduced or complete insensitivity to exogenous ethylene. The other class of mutants, the constitutive hormone response mutants, display constitutive ethylene response phenotypes in the absence of exogenously applied hormones.

The first component of the ethylene signal transduction pathway to be identified was an ethylene receptor gene from Arabidopsis, ETR1. This gene encodes a histidine kinase with homology to bacterial two-component systems (Chang et al., Science 262:539-544 (1993)).

To date, a total of five ethylene receptor genes have been cloned from Arabidopsis, ETR1, ETR2, ERS1, ERS2, and EIN4 (Hua et al., Science 269:1712-1714 (1995); Hua et al., Plant Cell 10:1321-1332 (1998); Sakai et al., Proc. Natl. Acad. Sci. USA 95:5812-5817 (1998)). Loss-of-function mutations in multiple ethylene receptors increase sensitivity to ethylene, indicating that the receptors are negative regulators of ethylene response (Hua et al., Cell 94:261-271 (1998)).

Downstream components of the pathway have also been identified in Arabidopsis. These include CTR1 (constitutive triple response), which is homologous to the Raf family of serine/threonine kinases (Kieber et al., Cell 72: 427-441 (1993)), and which interacts with the two ethylene receptor proteins ETR1 (ethylene receptor1) and ERS1 in a yeast two-hybrid system (Clark et al., Proc. Natl. Acad. Sci. USA 95:5401-5406 (1998)). Loss-of-function mutations in CTR1 cause a constitutive ethylene signaling phenotype of severe leaf epinasty and reduced leaf expansion, indicating that, like the receptor proteins, CTR1 is a negative regulator of ethylene response.

EIN2 is an additional component of the ethylene signal transduction pathway. Epistasis analyses indicate that EIN2 acts downstream of CTR1. EIN2 is an integral membrane protein with 12 membrane spanning regions and is homologous to the Nramp metal-ion transport proteins (Alonso et al., Science 284:2148-2152 (1999)). EIN2 loss-of-function mutants are insensitive to ethylene, indicating that these proteins are positive regulators of ethylene response.

Several transcription factors that control the expression of ethylene-regulated genes have also been identified. The Arabidopsis EIN3 family contains several proteins that bind to an ethylene response element in the promoter of the Ethylene Response Factor 1 (ERF1) gene. ERF1 binds to the promoters of pathogenesis-related (PR) genes and regulates their expression (Solano et al., Genes & Development 12:3703-3714 (1998)). Loss-of-function mutations in EIN3 reduce sensitivity to ethylene. Over-expression of wild-type EIN3 results in constitutive ethylene response (Chao et al., Cell 89:1133-1144 (1997)). Therefore, EIN3 is a positive regulator of ethylene response.

Isolation of several components of the ethylene signal transduction pathway has made it possible to genetically engineer crop species with altered ethylene sensitivity. In tomato, antisense expression of the tomato ethylene receptor LeETR4 resulted in constitutive ethylene responses, including leaf epinasty, premature flower senescence, and accelerated fruit ripening (Tieman et al., Proc. Natl. Acad. Sci. 97:5663-5668 (2000)). Over-expression of the tomato ethylene receptor protein NR resulted in tomato plants with reduced ethylene sensitivity as evidenced by increased stem and seedling elongation and reduced necrosis in response to a bacterial pathogen (Ciardi et al., Plant Phys. 123:81-92 (2000)). Heterologous expression of mutant receptor proteins can also decrease sensitivity. For example, over-expression of the mutant Arabidopsis ethylene receptor protein ETR1-1 in both petunia and tomato resulted in delayed flower abscission, flower senescence, and fruit ripening (Wilkinson et al., Nature Biotech. 15:444-447 (1997)).

Nevertheless, until the present invention here has remained a need for an understanding of the two ethylene-regulated developmental processes in cotton: flower and leaf abscission. Premature flower abscission can greatly reduce boll set in cotton, with a potential reduction in total yield. In contrast, premature leaf abscission in cotton is also a desirable trait, thereby eliminating the need for chemical treatments to reduce leaf cover before harvest. Thus, there is also a need for a transgenic plant having reduced ethylene sensitivity in flowers (to prevent flower abscission), but with increased ethylene sensitivity in leaves (to induce leaf abscission).

### SUMMARY OF THE INVENTION

The present invention is directed to methods and compositions for controlled regulation of leaf and/or flower abscission in transformed plants. In a preferred embodiment flower abscission is reduced, while in another preferred embodiment leaf abscission is induced or accelerated (premature) as compared with untreated wild type plants. In yet another preferred embodiment, both flower and leaf abscission is modulated, and the method comprises overexpressing EIN2 and EIN3. In a particularly preferred embodiment, the methods comprise overexpression of the EIN2/EIN3 genes in a selected transgenic plant, which is cotton.

1 In at least one embodiment of the invention, the overexpression of the EIN2 gene is driven or controlled by a flower abscission zone-specific promoter. In another embodiment, the overexpression of the EIN3 gene is driven or controlled by an inducible promoter. In yet another embodiment in which EIN2 and EIN3 are both overexpressed, the overexpression of the EIN2 gene is driven or controlled by a flower abscission zone-specific promoter and the overexpression of the EIN3 gene is driven or controlled by an inducible promoter.

The invention further provides a method for producing at least one cell line in which EIN2/EIN3 is overexpressed comprising: transforming a first cotton plant tissue with an exogenous EIN2 gene or active fragment thereof, selected to provide overexpression of EIN2; and transforming a second cotton plant tissue with an exogenous EIN3 gene or active fragment thereof, selected to provide inducible overexpression of EIN3; then genetically crossing the EIN2 and EIN3 overexpressing transformed cell lines; and selecting at least one crossed EIN2/EIN3 overexpressing cell line displaying a strong phenotype of combined reduced flower abscission and inducible premature leaf abscission. In a preferred embodiment the crossed EIN2/EIN3 overexpressing cell line is in cotton.

Also provided is a transformed plant or plant part, in which the cells, organs, flowers, tissues, seeds or progeny, comprise the EIN2 overexpressing gene or active fragment thereof; the EIN3 overexpressing gene or active fragment thereof; or more preferably the combined EIN2 and EIN3 overexpressing genes or active fragments thereof. In addition, a cotton cell line produced by the foregoing methods is provided. The transformed plant or cell line is further provided, wherein the genes or active fragments thereof, comprise recombinant nucleic acids.

Further provided is a transformed plant or plant part, in which the cells, organs, flowers, tissues, seeds or progeny comprise the controlled overexpression of EIN2 or EIN3 polypeptides or combined EIN2/EIN3 polypeptides encoded by EIN2 or EIN3 genes or active fragments thereof, or by combined EIN2/EIN3 genes or active fragments thereof.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE FIGURES

FIG. 1 is a photograph showing delayed flower senescence in two independent lines (EIN2-1 and EIN2-2) of EIN2 co-suppressed tomato plants and a wild type plant as labeled. Flower clusters were cut from the plant, placed in a vial of water, and treated with 10 ppm ethylene for 16 hrs. Flowers are shown 48 hrs after ethylene treatment.

FIG. 2 is a photograph of a gel showing overexpression of the TGV transcription factor in leaves of transgenic tomato plants. The chimeric transcription factor TGV was overexpressed in tomato under transcriptional control of a constitutive promoter. TGV expression was quantified by RT PCR in To plants from 10 independent transgenic lines.

### DETAILED DESCRIPTION OF THE INVENTION

The present invention is based upon the tissue specific manipulation of several ethylene-signaling genes, permitting the controlled regulation of leaf and/or flower abscission. As a result of the present invention, new insights into the mechanisms involved in the ethylene signaling pathway are evident.

In wild type plants, ethylene binding to its receptor inactivates the activity of ethylene receptors (presumably causing a reduction in the histidine kinase activity), and consequently causing induction of the ethylene response through activation (de-repression) of the signaling pathway. Loss-of-function ethylene receptor mutants have been shown to function as negative regulators of the signaling pathway and show significant functional overlap. Moreover, binding of ethylene to the receptor(s) presumably inhibits biochemical activity.

A number of biological stresses are known to induce ethylene production in plants, including wounding, abscission, bacterial, viral or fungal infection, and treatment with elicitors, such as glycopeptide elicitor preparations from fungal pathogen cells. In the case of abscission, a particular layer of cells in a zone located between the base of the leaf stalk and the stem (the abscission zone) responds to a complex combination of ethylene and other endogenous plant growth regulators by a process that is, to date, not fully understood. However, the effect of abscission is the controlled loss of part of the plant, typically localized; the result of which is visible as a dead leaf or flower, or as softening or “ripening” of fruit, ultimately leaving the plant wounded at the point of separation.

By “plant,” as used herein, is meant any whole plant, or any part thereof, of wild type, treated, genetically manipulated or recombinant plant or plant part, including transgenic plants. The term broadly refers to any and all parts of the plant, including plant cell, tissue, flower, leaf, stem, root, organ, and the like, and also including seeds, progeny and the like, whether such part is specifically named or not.

The inventors' cotton research program had two major goals: to reduce early-season flower abscission and accelerate late-season leaf abscission. Abscission of cotton flower buds (“squares”) is caused by biotic and abiotic stress, and can significantly reduce cotton yields. Leaf abscission is necessary to clear the cotton plant of debris before mechanical harvesting. This is currently induced by herbicide application. Since cotton flower and leaf abscission are positively regulated by the plant hormone ethylene, these responses are, in a preferred embodiment, manipulated by altering expression of ethylene signaling genes.

Square abscission is reduced through co-suppression of the cotton EIN2 gene. Promoters were evaluated for driving EIN2 expression, e.g., a constitutive promoter and a flower pedicel specific promoter from a cotton chitinase gene, although the invention is not intended to be so limited. Other promoters suitable for this purpose would also be known to one of skill in the art, and their use is encompassed by the present invention.

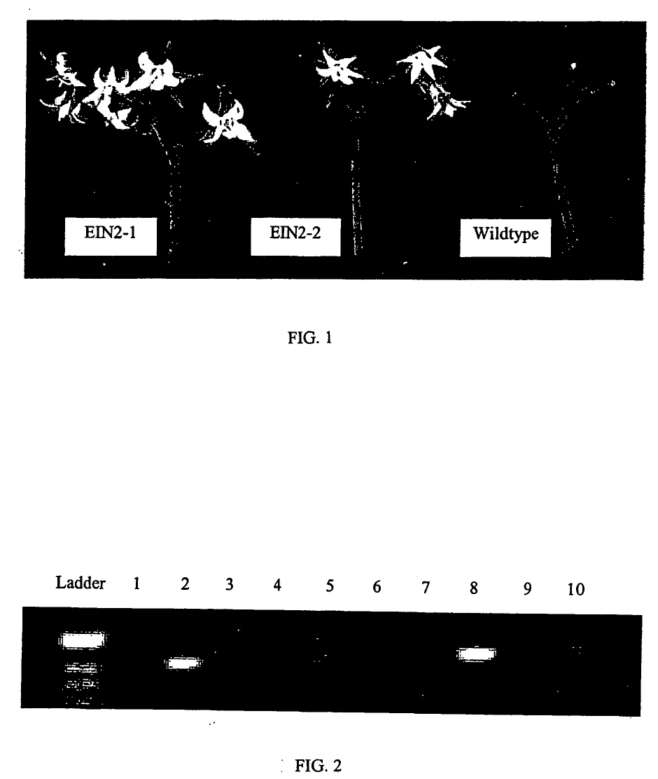

Preliminary work was conducted in tomato to test the efficacy of these approaches. Constitutive over-expression of the tomato EIN2 gene was found to effectively delay flower abscission by at least five-fold (5×) over that of normal wild-type plants. For example, wild-type tomato flowers treated with ethylene abscised 24 hours after treatment, while the transgenic flowers remained attached to the pedicel for at least 5 days after treatment (see FIG. 1, and as disclosed in greater detail in the examples that follow). Moreover, when progeny of the primary transformants were grown, it was confirmed that the controlled, delayed flower abscission trait is heritable in subsequent generations.

Consequently, to extend this work into cotton. The cotton EIN2 gene was cloned and the clones sequenced, and the construct for constitutive co-suppression was assembled and transformed into Agrobacterium. The cotton chitinase promoter, when evaluated in tomato, has been shown to drive pedicel-specific expression of a marker gene.

Cotton leaf abscission was accelerated through over-expression of the Arabidopsis EIN3 (AtEIN3) gene. Plants were developed in which chemically-inducible promoters are evaluated for their ability to drive AtEIN3 expression. By determining the optimal expression pattern for AtEIN3, developmentally-regulated promoters are selected, although one of ordinary skill in the art could adapt other promoters for this purpose by following the examples that follow herein below.

Since one of the promoters is inducible by ethanol, leaf abscission is accelerated in such plants by ethanol treatment, it became possible to control leaf removal from the plants by less expensive compositions, using methods in the field that were less toxic to the plants and the environment than herbicides.

Using a tomato model system, a glucocorticoid-inducible gene system was developed for regulating leaf abscission. In plants produced in which a glucocorticoid-inducible transcription factor (such as, but not limited to, TGV) was over-expressed (FIG. 2), no negative effects on plant growth and/or development was detected. Screening of these plants by recognized RT-PCR techniques, identified a series of transgenic lines having high levels of overexpression, which when crossed with a second set of transgenic plants containing the AtEIN3 gene under transcriptional control of a glucocorticoid-inducible promoter, demonstrate the controlled regulation of leaf abscission in accordance with a preferred embodiment of the invention. In an alternative embodiment, the construct comprises the transcription factor and AtEIN3 over-expression cassette on the same TDNA

In sum, preferred embodiments of the invention should be construed to include nucleic acid comprising isolated EIN2 having approximately 70% identity to Arabidopsis EIN2, which when placed under the control of a promoter acceptable to the selected plant species, results in the over-expression of EIN2 in the selected plant species as demonstrated by delayed flower abscission. Also included is nucleic acid embodiment comprising isolated Arabidopsis EIN3, which when placed under the control of a promoter acceptable to the selected plant species, results in the over-expression of EIN3 in the selected plant species as demonstrated by inducible leaf abscission.

In another preferred embodiment, the resulting plant lines are crossed, resulting in the combined effect of the over-expression of both EIN2 and EIN3, results in plants having delayed flower abscission and inducible leaf abscission. Further included within the present invention is any mutant, derivative, or homologue of the foregoing, or fragment thereof, which encodes the regulated EIN2-controlled delayed flower abscission or the regulated EIN3-controlled inducible leaf abscission, or in a preferred embodiment, the combination thereof.

In accordance with the present invention, nucleic acid sequences include, but are not limited to DNA, including and not limited to cDNA and genomic DNA; RNA, including and not limited to mRNA and tRNA, and may include chiral or mixed molecules.

Preferred nucleic acid sequences include, for example, those set forth in cotton EIN2 SEQUENCE ID NO: 1, as well as modifications in those nucleic acid sequences, including alterations, insertions, deletions, mutations, homologues and fragments thereof encoding a regulatory protein, EIN2 or EIN3, or a combination of regulatory proteins, EIN2/EIN3, in the ethylene response pathway resulting in plants exhibiting EIN2-controlled delayed flower abscission and/or regulated EIN3-controlled inducible leaf abscission.

A “fragment” of a nucleic acid is included within the present invention if it encodes substantially the same expression product as the isolated nucleic acid, or if it encodes peptide(s) disclosed herein having the desired regulatory effect(s) on flower and/or leaf abscission.

The invention should also be construed to include peptides, polypeptides or proteins comprising EIN2 and/or EIN3, alone or in EIN2/EIN3 combination, or any mutant, derivative, variant, analog, homologue or fragment thereof, having flower and/or leaf abscission controlling activity in the ethylene signaling pathway. The terms “protein,” “peptide,” “polypeptide,” and “protein sequences” are used interchangeably within the scope of the present invention, and include, but are not limited to the amino acid sequences corresponding to nucleic acid SEQUENCE ID NO: 1, as well as those sequences representing mutations, derivatives, analogs or homologues or fragments thereof having having flower and/or leaf abscission controlling activity in the ethylene response pathway.

The invention also provides for analogs of proteins, peptides or polypeptides encoded by EIN2 or EIN3, or the combination EIN2/EIN3. “Analogs” can differ from naturally occurring proteins or peptides by conservative amino acid sequence differences or by modifications which do not affect sequence, or by both. “Homologues” are chromosomal DNA carrying the same genetic loci; when carried on a diploid cell there is a copy of the homologue from each parent.

For example, conservative amino acid changes may be made, which although they alter the primary sequence of the peptide, do not normally alter its function. Conservative amino acid substitutions of this type are known in the art, e.g., changes within the following groups: glycine and alanine; valine, isoleucine and leucine; aspartic acid and glutamic acid; asparagine and glutamine; serine and threonine; lysine and arginine; or phenylalanine and tyrosine. Modifications (which do not normally affect the primary sequence) include in vivo or in vitro chemical derivatization of the peptide, e.g., acetylation or carbonation. Also included are modification of glycosylation, e.g., modifications made to the glycosylation pattern of a polypeptide during its synthesis and processing, or further processing steps. Also included are sequences in which amino acid residues are phosphorylated, e.g., phosphotyrosine, phosphoserine or phosphothreonine.

Also included in the invention are polypeptide embodiments which have been modified using ordinary molecular biology techniques to improve their resistance to proteolytic degradation or to optimize solubility or to render them more effective as a regulatory agent. Analogs of such peptides include those containing residues other than the naturally occurring L-amino acids, e.g., D-amino acids or non-naturally occurring synthetic molecules. However, the polypeptides of the present invention are not intended to be limited to products of any specific exemplary process defined herein.

“Derivative” is intended to include both functional and chemical derivatives, including fragments, segments, variants or analogs of a molecule. A molecule is a “chemical derivative” of another, if it contains additional chemical moieties not normally a part of the molecule. Such moieties may improve the molecule's solubility, absorption, biological half-life, and the like, or they may decrease toxicity of the molecule, eliminate or attenuate any undesirable side effect of the molecule, and the like. Moieties capable of mediating such effects are disclosed, for example, in Remington's Pharmaceutical Sciences (1980). Procedures for coupling such moieties to a molecule are well known in the art. Included within the meaning of the term “derivative,” as used in the present invention, are “alterations,” “insertions,” and “deletions” of nucleotides or peptides, polypeptides or the like.

A “variant” or “allelic or species variant” of a protein refers to a molecule substantially similar in structure and biological activity to the protein. Thus, if two molecules possess a common activity and may substitute for each other, it is intended that they are “variants,” even if the composition or secondary, tertiary, or quaternary structure of one of the molecules is not identical to that found in the other, or if the amino acid or nucleotide sequence is not identical. A “fragment” of a polypeptide is included within the present invention if it retains substantially the same activity as the purified peptide, or if it has EIN2 or EIN3 or combined controlled EIN2/EIN3 activity resulting in delayed flower abscission along with inducible leaf abscission.

In accordance with the invention, the EIN2 or EIN3 nucleic acid sequences employed in certain embodiments may be exogenous sequences. Exogenous or heterologous, as used herein, denotes a nucleic acid sequence which is not obtained from and would not normally form a part of the genetic makeup of the plant, cell, organ, flower or tissue to be transformed, in its untransformed state.

Transformed plant cells, tissues and the like, comprising nucleic acid sequences of EIN2 and/or EIN3, such as, but not limited to, the nucleic acid sequence of SEQUENCE ID NO: 1, are within the scope of the invention. Transformed cells of the invention may be prepared by employing standard transformation techniques and procedures, such as, but not limited to, those set forth in Sambrook et al., Molecular Cloning: A Laboratory Manual, 2nd ed., Cold Spring Harbor Laboratory Press, Cold Spring Harbor, N.Y. (1989).

By the term “nucleic acid encoding” the resulting plant cell and the like having controlled flower and/or leaf abscission activity, as used herein, is meant a gene encoding a polypeptide capable of controlling the abscission as described above. The term is meant to encompass DNA, RNA, and the like.

As described in the following Examples, EIN2 and EIN3 genes encode proteins having specific domains located therein, for example, terminal extensions, transmembrane spans, TM1 and TM2, nucleotide binding folds, a putative regulatory domain, and the C-terminus. A mutant, derivative, homologue or fragment of the subject gene is, therefore also one in which selected domains in the expressed protein share significant identity (at least about 40% identity to that of Arabidopsis) with the same domains in the preferred embodiment of the present invention. It will be appreciated that the definition of such a nucleic acid encompasses those gene(s) having at least about 40% identity to corresponding gene(s) in Arabidopsis, in any of the described domains contained therein.

In addition, when the term “identity” is used herein to refer to the domains of these proteins, it should be construed to be applied to identity at both the nucleic acid and the amino acid levels. Significant identity between similar domains in such nucleic acids or their protein products is considered to be at least about 40%, preferably the identity between domains is at least about 50%, more preferably, at least about 60%, even more preferably, at least about 70%, even more preferably, at least about 80%, yet more preferably, at least about 90% and most preferably the identity is about 99%, or in the protein expression products thereof.

According to the present invention, preferably, the isolated nucleic acid encoding the EIN2 and/or EIN3 polypeptide, or fragment thereof, is full length or of sufficient length to effect controlled regulation over flower and/or leaf abscission in resulting plant(s). In one embodiment the nucleic acid is at least about 1000 nucleotides in length. More preferably, it is at least 2000 nucleotides, even more preferably, at least 3500 nucleotides, yet more preferably, at least 4000 nucleotides, and even more preferably, at least 4900 nucleotides in length. In another embodiment, preferably, the putative or purified preparation of the isolated polypeptide(s) having abscission-controlling activity in the ethylene signal system is at least about 300 amino acids in length. More preferably, it is at least 500 amino acids, even more preferably, at least 1000 amino acids, yet more preferably, at least 1200 amino acids, and even more preferably, at least 1600 amino acids in length. In an additional embodiment the polypeptide encodes the full length EIN2 and/or EIN3 protein or a regulated combined EIN2/EIN3 version thereof.

The invention further includes a vector or vectors comprising a gene encoding EIN2 and/or EIN3. DNA molecules composed of a protein gene or a portion thereof, can be operably linked into an expression vector and introduced into a host cell to enable the expression of these proteins by that cell. Alternatively, a protein may be cloned in viral hosts by introducing a “hybrid” gene operably linked to a promoter into the viral genome. The protein may then be expressed by replicating such a recombinant virus in a susceptible host. A DNA sequence encoding a protein molecule may be recombined with vector DNA in accordance with conventional techniques. When expressing the protein molecule in a virus, the hybrid gene may be introduced into the viral genome by techniques well known in the art.

Thus, embodiments of the present invention encompass the expression of the desired proteins in either prokaryotic or eukaryotic cells, or viruses, which replicate in prokaryotic or eukaryotic cells. Procedures for generating a vector for delivering the isolated nucleic acid or a fragment thereof, are well known, and are described for example in Sambrook et al., supra. Suitable vectors include, but are not limited to, disarmed Agrobacterium tumor inducing (Ti) plasmids (e.g., pBIN19) containing a target gene under the control of a vector, such as the cauliflower mosaic (CaMV) 35S promoter (Lagrimini et al, Plant Cell 2:7-18 (1990)) or its endogenous promoter (Bevan, Nucl. Acids Res. 12:8711-8721(1984)), tobacco mosaic virus and the like.

Once the vector or DNA sequence containing the constructs has been prepared for expression, the DNA constructs may be introduced or transformed into an appropriate host. Various techniques may be employed, such as protoplast fusion, calcium phosphate precipitation, electroporation, or other conventional techniques. As is well known, viral sequences containing a “hybrid” protein gene may be directly transformed into a susceptible host, or first packaged into a viral particle, and then introduced into a susceptible host by infection. After the cells have been transformed with the recombinant DNA (or RNA) molecule, or the virus or its genetic sequence is introduced into a susceptible host, the cells are grown in media and screened for appropriate activities. Expression of the sequence results in the production of the protein of preferred embodiments of the present invention.

Procedures for generating a plant cell, tissue, flower, organ or a fragment thereof, are well known in the art, and are described, for example, in Sambrook et al., supra. Suitable cells include, but are not limited to, cells from yeast, bacteria, mammal, baculovirus-infected insect, and plants, with applications either in vivo, or in tissue culture. Also included are plant cells transformed with the gene of interest for the purpose of producing cells and regenerating plants having modulated flower and/or leaf abscission capability. Suitable vector and plant combinations will be readily apparent to those skilled in the art and can be found, for example, in Maliga et al., 1994, Methods in Plant Molecular Biology: A Laboratory Manual, Cold Spring Harbor, N.Y.).

Transformation of plants may be accomplished, e.g., using Agrobacterium-mediated leaf disc transformation methods described by Horsch et al., 1988, Leaf Disc Transformation: Plant Molecular Biology Manual) or other methods known in the art. Numerous procedures are known in the art to assess whether a transgenic plant comprises the desired DNA, and need not be reiterated at this point.

The expression of the desired protein in eukaryotic hosts requires the use of eukaryotic regulatory regions. Such regions will, in general, include a promoter region sufficient to direct the initiation of RNA synthesis. Preferred eukaryotic promoters include, but are not limited to, the SV40 early promoter (Benoist et al., Nature (London) 290:304-310 (1981)); the yeast gal4 gene promoter (Johnston et al., Proc. Natl. Acad. Sci. (USA) 79:6971-6975 (1982)) and the exemplified pYES3 PGK1 promoter. In addition, inducible promoters are used as described below. As is widely known, translation of eukaryotic mRNA is initiated at the codon encoding the first methionine. For this reason, it is preferable to ensure that the linkage between a eukaryotic promoter and a DNA sequence which encodes the desired protein does not contain any intervening codons which are capable of encoding a methionine (i.e., AUG).

The desired protein encoding sequence and one or more operably linked promoters may be introduced into a recipient prokaryotic or eukaryotic cell either as a non-replicating DNA (or RNA) molecule, which may either be a linear molecule or, more preferably, a closed covalent circular molecule. Since such molecules are incapable of autonomous replication, the expression of the desired protein may occur through the transient expression of the introduced sequence. Alternatively, permanent expression may occur through the integration of the introduced sequence into the host chromosome. For expression of the desired protein in a virus or plant, the hybrid gene operably linked to a promoter is typically integrated into the viral genome, be it RNA or DNA. Cloning into plants is well known and thus, one of skill in the art will know numerous techniques to accomplish such cloning.

Cells which have stably integrated the introduced DNA into their chromosomes can be selected by also introducing one or more reporter genes or markers which allow for selection of host cells which contain the expression vector. The reporter gene or marker, such as kanamycin resistance, may complement an auxotrophy in the host (such as leu2, or ura3, which are common yeast auxotrophic markers), biocide resistance, e.g., antibiotics, or the effect can be seen as a physical response, such as flower or leaf abscission or the like. A selectable marker gene, such as kanamycin resistance, can either be directly linked to the DNA gene sequences to be expressed, or introduced into the same cell by co-transfection.

Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol. 3:280 (1983), and others.

In another embodiment, the introduced sequence is incorporated into a plasmid or viral vector capable of autonomous replication in the recipient host cell. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

The invention further defines methods for manipulating the nucleic acid in a plant to permit the regulation, control or modulation of abscission, flower or leaf senescence, flower maturation, fruit ripening, or response to stress. In a preferred embodiment the method initiates or enhances the above responses; whereas, in another preferred embodiment the method inhibits or prevents the above responses.

Thus, methods of the present invention define embodiments in which controlled flower abscission activity is prevented or inhibited. By “prevention” is meant the cessation of flower drop for a period of time beyond which ethylene pathway-controlled flower abscission normally occurs in the selected plant species. By “inhibition” is meant a statistically significant reduction in flower abscission activity, as compared with plants, plant cells, organs, flowers or tissues grown without the inhibitor or disclosed method of inhibition. Preferably, the inhibitor reduces flower abscission by at least 20%, more preferably by at least 50%, even more preferably by 80% or greater, and also preferably, in a dose-dependent manner. Once inhibitors satisfying these requirements are identified, the utilization of assay procedures to identify the manner in which flower abscission is inhibited are particularly useful.

Similarly, methods of the present invention are defined in which leaf abscission activity is “induced,” “initiated,” “stimulated” or “enhanced” if there is a statistically significant increase in the amount of controlled leaf abscission activity, as compared with plants, plant cells, organs, flowers or tissues grown without the enhancer or disclosed method of enhancement. Preferably, the enhancer increases controlled leaf abscission by at least 20%, more preferably by at least 50%, even more preferably by 80% or greater, and also preferably, in a dose-dependent manner. Once enhancers satisfying these requirements are identified, the utilization of assay procedures to identify the manner in which controlled leaf abscission is enhanced are particularly useful.

In a preferred embodiment of the invention the enhanced leaf abscission is under the control of an inducing composition. For example, encompassed are EIN3 cDNA sense and antisense constructs, wherein inducible promoters, such as a glucocorticoid-inducible promoter (Bohner et al., Plant J. 19:87-95 (1999)) or an ethanol-inducible promoter (Salter et al., Plant J. 16:127-132 (1998)), or an ecdysone agonist-inducible promoter (Martinez et al., Plant J. 19:97-106 (1999)) or others, are incorporated to regulate the expression of the gene. Over-expression of EIN3 in Arabidopsis was shown to induce several ethylene responses, including inhibition of leaf expansion. Since growth was severely reduced in EIN3 over-expressing Arabidopsis lines, an inducible promoter was used to prevent undesirable pleiotropic effects of the transgene. When leaves were treated, for example in the glucocorticoid inducible promoter model, treatment involved the application of a solution of glucocorticoid dexamethasone, and the result was severe leaf epinasty of the transgenic plants.

A selected glucocorticoid-inducible transcription factor was TGV, which is essential for the operation of EIN3. TGV (a chimeric gene comprising a tetracylene repressor, a glucocorticoid receptor, and the transcriptional activator VP16) allows chemical induction of EIN3 expression, which in turn accelerates leaf abscission. The DNA samples from 10 independent transgenic lines (a portion of the TGV gene that was amplified from RNA isolated from leaves of the transgenic tomato plants by RT PCR), were run in a 1% agarose gel in TBE buffer at 120 V for 1 hr as shown in FIG. 2. The ladder is a 1 kb DNA ladder from Invitrogen (Carlsbad, Calif.).

Similar inducible effects were seen for the other constructs, such as when the selected transgenic plants were sprayed with ethanol solution or the ecdysone agonist, muristeroneA. However, for cost and efficiency purposes, the ethanol-inducer is a good selection for field use. By comparison, treatment of wild type plants with each of the chemical inducers had no visible effects on growth or development.

Selected embodiments of the invention further contain constructs comprising other regulatory genes in a sense or antisense direction, in addition to EIN2 and/or EIN3, alone or in EIN2/EIN3 combination. For example, when the constructs further contained an antisense copy of CTR1 (a negative regulator of ethylene response), the transcriptional control of the inducible promoters, the ethylene response and resulting leaf abscission was as previously noted. However, the effect of treatment of the CTR1 antisense plants with dexamethasone, ethanol, and muristeroneA resulted in a rate of leaf abscission that was less rapid as had been seen in those plants in which EIN3 was over-expressed.

The invention further features an isolated preparation of a nucleic acid that is antisense in orientation to a portion or all of a plant gene, such as is described for constructs comprising the antisense CTR1 gene. The antisense nucleic acid should be of sufficient length as to inhibit expression of the target gene of interest. The actual length of the nucleic acid may vary, depending on the target gene, and the region targeted within the gene. Typically, such a preparation will be at least about 15 contiguous nucleotides, more typically at least 50 or even more than 500 contiguous nucleotides in length.

As used herein, a sequence of nucleic acid is considered to be antisense when the sequence being expressed is complementary to, and essentially identical to the non-coding DNA strand of the selected gene, but which does not encode the expression product of the gene, such as CTR1. “Complementary” refers to the subunit complementarity between two nucleic acids, e.g., two DNA molecules. When a nucleotide position in both molecules is occupied by nucleotides normally capable of base pairing with each other, then the nucleic acids are said to be complementary to each other. Thus two nucleic acids are complementary when a substantial number (at least 50%) of the corresponding positions in each of the molecules are occupied by nucleotides which normally base-pair with each other (e.g., A:T and G:C nucleotide pairs).

By “transgenic plant” as used herein, is meant a plant, plant cell, tissue, flower, organ, including seeds, progeny and the like, or any part of a plant, which comprise a gene inserted therein, which gene has been manipulated to be inserted into the plant cell by recombinant DNA technology. The manipulated gene is designated a “transgene.” The “nontransgenic,” but substantially homozygous “wild type plant,” as used herein, means a nontransgenic plant from which the transgenic plant was generated. The transgenic transcription product may also be oriented in an antisense direction as describe above.

The generation of transgenic plants comprising modified or exogenous sense or antisense DNA encoding EIN2 and/or EIN3 of the ethylene signaling pathway, may be accomplished by transforming the plant with a plasmid, liposome, or other vector encoding the desired DNA sequence. Such vectors would, as described above, include, but are not limited to the disarmed Agrobacterium tumor-inducing (Ti) plasmids containing a sense or antisense strand placed under the control of a strong constitutive promoter, such as 35CaMV 35S or under an inducible promoter. Methods of generating such constructs, plant transformation and plant regeneration methods are well known in the art once the sequence of the gene of interest is known, for example as described in Ausubel et al., 1993, Current Protocols in Molecular Biology, Greene & Wiley, New York).

In accordance with the present invention, plants included within its scope include both higher and lower plants of the Plant Kingdom. Mature plants, including rosette stage plants, and seedlings are included in the scope of the invention. A mature plant, therefore, includes a plant at any stage in development beyond the seedling. A seedling is a very young, immature plant in the early stages of development. Transgenic plants are also included within the scope of the present invention, having a phenotype characterized by EIN2- and/or EIN3-controlled flower and/or leaf abscission. Preferred plants of the present invention include, but are not limited to, high yield crop species for which cultivation practices have already been perfected (including monocots and dicots), or engineered endemic species.

Preferred plants in which the EIN2-control of flower abscission is exhibited include any commercially valuable or home-grown flowering species, e.g., roses, carnations, or chrysanthemums, and many others, or leafy ornamental plants, such a geranium and many others. Preferred plants in which the EIN3-control of leaf abscission is exhibited include any commercially valuable or home-grown leafy green ornamental plant, such as Ficus, palms, and the like, in which longevity of the leaf stem on the plant (delayed abscission) is of particular relevance, as are harvested plants in which inducing premature leaf abscission facilitates mechanical or other means of harvesting. Delayed flowering in such plants may also be advantageous. However, a particularly preferred advantage of the present invention is seen in plants under the combined control of EIN2/EIN3, particularly including commercially valuable flowering plants, such as cotton and the like, in which longevity of the flower on the stem (delayed abscission) is of particular relevance, e.g., harvested flowers or flower parts, such as in food crops, e.g., broccoli, cauliflower, etc. or certain flowering herbs or spices, and wherein harvesting, such as by a mechanical harvester, is facilitated by the early removal of plant leaves and plant debris by inducing premature leaf abscission.

The present invention is further described in the following examples. These examples are not to be construed as limiting the scope of the appended claims.

### EXAMPLES

Initial work was done in tomato to evaluate the effectiveness of each construct. Once the construct was proven to be effective, the experiment was repeated in cotton using an analogous construct.

Regulation of EIN2

Plant material. Tomato (Lycopersicon esculentum cv. Pearson) and cotton (Gossypium hirsutum cv. Coker 312) plants were grown under standard greenhouse conditions. Flower and leaf tissue for RNA extraction was harvested, frozen in liquid nitrogen, and stored at −80° C.

Isolation of the cotton EIN2 genomic clone. The Arabidopsis EIN2 cDNA was used to screen a cotton genomic library. Several cotton genomic clones were isolated, from which HindIII and BamHI fragments were subcloned and sequenced. Coding regions from one of the clones was found to have 70% nucleotide identity with the Arabidopsis EIN2 cDNA. This clone was used for transformation of cotton.

Delay of flower abscission in tomato. Flower abscission was delayed in tomato by manipulating EIN2 gene expression. Transgenic plants over-expressing a 2.1 kb partial cDNA encoding the 3′ end of the EIN2 gene were produced through Agrobacterium-mediated transformation. This partial E1N2 cDNA was under transcriptional control of either the constitutive figwort mosaic virus promoter (Richins et al., Nucleic Acid Res. 15: 8451-8466 (1987)) or an abscission zone specific promoter from a cotton pathogenesis-related gene. Primary transformants were self-pollinated and lines that were homozygous for the transgene were selected from subsequent generations.

Production of transgenic plants. Transgenic tomato (Lycopersicon esculentum cv. Pearson) and cotton (Gossypium hirsutum cv. Coker 312) were produced using Agrobacterium-mediated transformation with kanamycin resistance as a selectable marker. Introduction and inheritance of the transgenes was confirmed by PCR using primers specific for the selectable marker or, for the crosses, primers specific to each transgene. All experiments were performed on plants that were homozygous for the transgene.

RNA isolation. Total RNA was isolated, as previously described by Ciardi et al., 2000. For real-time quantitative PCR, RNA samples were treated with DNase I (Ambion, Austin, Tex.) followed by purification with a RNeasy RNA extraction kit (Qiagen, Valencia, Calif.).

Lines with Delayed Flower Abscission. To identify lines with delayed flower abscission, flowers were tagged on the day of anthesis, and the number of days until abscission was recorded. Both the constitutive promoter construct and the abscission specific construct had similar effects on flower longevity. Unfertilized wildtype flowers abscised an average of 4 days after anthesis, while many of the transgenic lines still contained turgid unfertilized flowers for more than 20 days after anthesis. Fertilized wildtype flowers also abscised from the developing fruit an average of 4 days after anthesis, while flowers on the EIN2 sense lines remained attached to the fruit for at least 21 days after anthesis (FIG. 1)

Based upon quantitative real-time PCR, transgenic lines with the greatest flower longevity also exhibited the lowest EIN2 expression levels in flowers, indicating co-suppression of the native EIN2 gene.

Regulation of EIN3

Induction of leaf abscission in tomato. Six different constructs were evaluated for the promotion of premature leaf abscission in tomato. The first construct contained the Arabidopsis EIN3 cDNA in sense orientation under control of a glucocorticoid-inducible promoter (Bohner et al., Plant J. 19:87-95 (1999)). The second and third constructs contained the same Arabidopsis EIN3 cDNA under control of an ethanol-inducible promoter (Salter et al., Plant J. 16:127-132 (1998)), or an ecdysone agonist-inducible promoter (Martinez et al., Plant J. 19:97-106 (1999)). Over-expression of EIN3 in Arabidopsis was shown to induce several ethylene responses, including inhibition of leaf expansion. Since growth was severely reduced in EIN3 over-expressing Arabidopsis lines, an inducible promoter was used in tomato to prevent undesirable pleiotropic effects of the transgene.

Homozygous EIN3 lines were isolated as described above for EIN2, and are then evaluated for inducible leaf abscission. Leaves are treated with the synthetic glucocorticoid dexamethasone by painting the upper surface of each leaf with a 10 mg/L solution. Dexainethasone treatment of the transgenic plants results in severe leaf epinasty of the transgenic plants and leaf abscission rates ranging from 2 to 4 days after treatment. Similar effects are seen for the other constructs when the plants were sprayed with a 10% ethanol solution or a 1.5 mM solution of the ecdysone agonist muristeroneA. Treatment of wildtype plants with each of the chemical inducers had no visible effects on growth and development.

Three additional constructs are assembled containing an antisense copy of TCTR1, the tomato homologue of the Arabidopsis CTR1 gene, under transcriptional control of the three promoters mentioned above. Since CTR1 is a negative regulator of ethylene response, antisense expression of CTR1 would be expected to increase ethylene response. Leaf abscission rates are evaluated by the methods described above.

Although treatment of TCTR1 antisense plants with dexamethasone, ethanol, and muristeroneA also induced leaf abscission, abscission was not as rapid as it was for those plants in which EIN3 was over-expressed, wherein abscission occurred 5 to 7 days after treatment. Since ethanol was the least expensive and least toxic of the three chemical inducers, plants containing the ethanol-inducible promoter were the most easily adaptable to field production. Therefore, the plants over-expressing EIN3 that contained the ethanol-inducible promoter were the focus of further evaluation.

Combined Regulation of EIN2 and EIN3

Combining delayed flower abscission and accelerated leaf abscission. To produce plants with delayed flower abscission and accelerated leaf abscission, the EIN2 over-expressing and EIN3 over-expressing tomato lines were crossed, and plants which were homozygous for both transgenes (EIN2/EIN3) were selected from subsequent generations. The resulting EIN2/EIN3 over-expressing plants maintain the characteristics of each line, and exhibit delayed flower abscission along with glucocorticoid-inducible leaf abscission.

Delayed flower abscission and accelerated leaf abscission in cotton. Since combined EIN2/EIN3 over-expression was effective in delaying flower abscission and promoting leaf abscission in tomato, similar constructs are used in cotton. A 4.9 kb genomic clone was isolated from a cotton genomic library. It contains approximately 2.5 kb of the cotton EIN2 coding region set forth in SEQUENCE ID NO:1, having 70% identity with the EIN2 gene of Arabidopsi, was used for over-expression to induce co-suppression. As in tomato, the Arabidopsis EIN3 gene is used for over-expression in cotton. Tissue specific cotton promoters are also used for each construct.

To delay flower abscission, a flower abscission zone specific promoter from a cotton chitinase gene was selected to drive expression of the cotton EIN2 gene. Similarly, a leaf senescence-inducible promoter from a cotton gene is selected to drive over-expression of EIN3 for induced leaf abscission. Cotton is transformed with each of these constructs and lines displaying the strongest phenotypes are selected. The EIN2 and EIN3 over-expressing cotton lines were then crossed and plants homozygous for both transgenes are evaluated. The EIN2/EIN3 over-expressing cotton lines exhibit greatly reduced flower abscission and premature leaf abscission. Constructs comprising the transcription factor and AtEIN3 over-expression cassette on the same TDNA are under current evaluation and will also be adapted into cotton.

Based upon the foregoing effectiveness of the controlled over-expression of EIN2/EIN3, independently in tomato and in cotton, to produce plant lines characterized by greatly reduced flower abscission, as well as induced premature leaf abscission, the methods and constructs of the present invention are shown to be applicable to any plant in which the disclosed characteristics are desired. That is, the disclosed methods and compositions are effective to produce any plant in which one wishes to cause EIN2-controlled reduction of flower abscission, or induce EIN3-controlled premature leaf abscission, or more preferably the combined EIN2/EIN3-controlled reduction of flower abscission along with induced premature leaf abscission.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art that the invention may be subject to various modification and additional embodiments, and that certain of the details described herein can be varied considerably without departing from the basic principles of the invention. Such modifications and additional embodiments are also intended to fall within the scope of the appended claims.

1. A method for controlling abscission in a transformed plant comprising effecting the overexpression of EIN2 or EIN3, or a combination thereof, in the plant, as compared with normal expression of the protein or proteins in a non-transformed matched wild type plant.

2. The method of claim 1, wherein flower abscission is modulated, and the method comprises overexpressing EIN2.

3. The method of claim 2, wherein flower abscission is inhibited or reduced.

4. The method of claim 1, wherein leaf abscission is modulated, and the method comprises overexpressing EIN3.

5. The method of claim 4, wherein leaf abscission is prematurely induced or accelerated.

6. The method of claim 1, wherein flower and leaf abscission is modulated, and the method comprises overexpressing EIN2 and EIN3.

7. The method of claim 1, wherein the transgenic plant is cotton.

8. The method of claim 1, further comprising driving overexpression of EIN2 by using a flower abscission zone-specific promoter to control EIN2 gene expression.

9. The method of claim 8, wherein the flower abscission zone-specific promoter comprises a cotton chitinase gene.

10. The method of claim 8, wherein the EIN2 gene is overexpressed, and wherein the EIN2 gene has at least 70% identity to the EIN2 gene in Arabidopsis.

11. The method of claim 10, wherein the overexpressed EIN2 gene is from cotton.

12. The method of claim 1, further comprising driving overexpression of EIN3 by using an inducible promoter to control EIN3 gene expression.

13. The method of claim 12, wherein the inducible promoter for inducing leaf abscission is selected from the group consisting of a glucocorticoid-inducible promoter, an ethanol-inducible promoter, and an ecdysone agonist-inducible promoter.

14. The method of claim 13, wherein the inducible promoter is an ethanol-inducible promoter.

15. A method of producing at least one cell line in which EIN2/EIN3 is overexpressed comprising:
transforming a first plant tissue with an exogenous EIN2 gene or active fragment thereof, selected to provide overexpression of EIN2; and
transforming a second plant tissue with an exogenous EIN3 gene or active fragment thereof, selected to provide inducible overexpression of EIN3; then
genetically crossing the EIN2 and EIN3 overexpressing transformed cell lines; and
selecting at least one resulting crossed EIN2/EIN3 overexpressing cell line displaying a strong phenotype of combined reduced flower abscission and inducible premature leaf abscission.

16. The method of claim 15, wherein the overexpressed EIN2 gene is from cotton.

17. The method of 15, wherein the crossed EIN2/EIN3 overexpressing cell line is in cotton.

18. The transformed plant or plant part, in which the cells, organs, flowers, tissues, seeds or progeny, comprise the overexpressed EIN2 gene or active fragment thereof according to the method of claim 8.

19. The transformed plant or plant part, in which the cells, organs, flowers, tissues, seeds or progeny, comprise the overexpressed EIN3 gene or active fragment thereof according to the method of claim 12.

20. The transformed plant or plant part, in which the cells, organs, flowers, tissues, seeds or progeny, comprise the overexpressed genes encoding EIN2 and EIN2 or active fragments thereof according to the method of claim 6.

21. A cotton cell line produced by the method of claim 16.

22. The transformed plant or plant part according to claim 20, wherein the genes or active fragments thereof, comprise recombinant nucleic acids.

23. The transformed plant or plant part, in which the cells, organs, flowers, tissues, seeds or progeny comprise the controlled overexpression of the combined EIN2/EIN3 polypeptides encoded by the EIN2/EIN3 genes or active fragments thereof, according to claim 15.

24. An isolated nucleic acid having at least 70% identity to Arabidopsis EIN2, and having EIN2 activity when overexpressed, resulting in the premature abscission of plant flowers or leaves when compared with wild type plants without EIN2 overexpression.
